(19) United States
(12) Patent Application Publication
(10) Pub. No.: US 2026/0347968 A1
(43) Pub. Date: Nov. 5, 2026

(54) ORGANIC MOLECULE COMPOSITIONS FOR LEACHING ORE CONTAINING METAL SULFIDES AND METHODS OF PREPARATION AND USE THEREOF

(71) Applicant: BASF SE, Ludwigshafen am Rhein (DE)

(72) Inventors: Julius Xaver Heidlas, Tucson, AZ (US); Mitchell Louis Catling, Tucson, AZ (US)

(73) Assignee: BASF SE, Ludwigshafen am Rhein (DE)

(21) Appl. No.: 19/111,550

(22) PCT Filed: Oct. 11, 2023
(86) PCT No.: PCT/US2023/076555

§ 371 Date: Mar. 13, 2025

Related U.S. Application Data
(60) Provisional application No. 63/416,418, filed on Oct. 14, 2022.

(51) Int. Cl. C22B 15/00; C22B 3/08; C22B 3/18; C25C 1/12

(52) U.S. Cl. CPC C22B 15/0071; C22B 3/08; C22B 3/18; C25C 1/12

(57) ABSTRACT

Disclosed herein are leaching solutions including a leaching additive having at least one organic compound having a sp3 hybridized sulfur and/or a sulfoxide; and a lixiviant. Also disclosed are methods of preparation and use thereof.

### FIELD

The disclosure relates generally to the field of mining. More particularly, the disclosure relates to compositions for leaching ore and methods of preparation and use thereof.

### BACKGROUND

The leaching of metal-sulfide minerals such as chalcopyrite has been well researched by academia and industry. Heap leaching, in particular, is used to extract precious metals, copper, uranium and other compounds from ore using a series of chemical reactions that assist in the dissolution of specific minerals. The main objective is to increase the rate and total recovery of a target metal from the ore in a typical heap leaching application at ambient temperature. Additives may be added to the leaching composition to help increase wetting and/or dissolution of the ore ultimately releasing more target metal. There is a need for improved leaching compositions to increase recovery of target metals from ore.

### BRIEF SUMMARY OF THE DRAWINGS

Reference will now be made in detail to the example embodiments of this disclosure, which are illustrated in the accompanying drawings. Wherever possible, the same reference numbers will be used throughout the drawings to refer to the same or like parts throughout the several views. Features of the various embodiments described herein may be combined with each other, unless specifically noted otherwise.

FIG. 1 shows a schematic diagram of an embodiment of a system for preparing and using a composition for leaching metal-sulfide-containing materials according to embodiments herein.

FIG. 2 shows a schematic diagram of a leach column setup used in Example 8.

FIG. 3 depicts the results of a leaching a mineral sulfide ore according to embodiments herein.

### BRIEF SUMMARY

Disclosed herein are various embodiments of a leaching solution comprising: a leaching additive containing an organic compound with at least one of a sp3 hybridized sulfur or a sulfoxide; and a lixiviant. In some embodiments, the leaching additive comprises one or more compound of Formula (A) or Formula (B):

R¹—S—R² (A)

R¹—(S═O)—R² (B)

wherein R¹ and R² is each, independently, selected from the below groups:
H, with the proviso that when either R¹ or R² is H, then R¹ and R² are not the same;
C₁ to C₁₀ linear or branched alkyl group which may be interrupted by one or more functional groups;
C₁ to C₁₀ hydroxyalkyl group, a ether thereof or an ester thereof;
C₁ to C₁₀ aminoalkyl group or a nitrogen functionalized derivative thereof, C₁ to C₁₀ carboxyalkyl;
an amino acid residue having the structure —(CₙH₂ₙ)CH(NH₂)COOH where n is an integer between 0 and 6;
C₂ to C₁₀ alkenyl group;
C₂ to C₁₀ alkynyl group;
a carbonyl group having the structure —C(═O)R³;
C₁ to C₁₀ alkoxy group;
C₂ to C₁₀ alkenyloxy group;
C₂ to C₁₀ alkynyloxy group;
C₂ to C₁₀ alkenylamino group;
C₂ to C₁₀ dialkenylamino; or
a sulfide group having the formula —S—R⁴,
wherein R³ and R⁴ is each, independently, H, OH, NH₂, C₁ to C₁₀ linear or branched alkyl group which may be interrupted by one or more functional groups, C₁ to C₁₀ hydroxyalkyl group, a ether thereof or an ester thereof, C₁ to C₁₀ aminoalkyl group or a nitrogen functionalized derivative thereof, C₁ to C₁₀ carboxyalkyl, an amino acid residue, C₂ to C₁₀ alkenyl group, C₂ to C₁₀ alkynyl group, a carbonyl group, C₁ to C₁₀ alkoxy group, C₂ to C₁₀ alkenyloxy group, C₂ to C₁₀ alkynyloxy group, C₂ to C₁₀ alkenylamino group, C₂ to C₁₀ dialkenylamino, or a sulfide group,
optionally, wherein Formula (A) or Formula (B) is cyclic.

In some embodiments, the leaching solution and/or leaching additive does not contain one or more of thiocarbonyl functionality or group, sulfonic acid functionality or group and/or an alkyl bridged polyamine. In embodiments, the leaching solution and/or leaching additive is free of one or more of thiourea, ethylene thiourea, formamidine disulfide, a sulfonamide, methane sulfonic acid, ethylenediamine, polyethylenimine, imidazole, diethylenetriamine, triethylenetetramine, tetraethylenepentamine, 1,2-diaminopropane, 2,3-butanediamine, or combinations thereof.

In some embodiments, the one or more leaching additive of Formula (A) or Formula (B) is cyclic. An example of a cyclic leaching additive is thioxane.

According to embodiments, the leaching solution can have a pH of less than about 4, less than about 3, less than about 2, or less than about 1, or about 0.1 to about 4.0, or about 1 to about 2.5, or any individual value or sub-range within these ranges. According to various embodiments, the leaching additive may be present in an amount of about 0.001 mM to about 67 mM, 0.1 mM to about 2 mM, or any individual value or sub-range within these ranges, based on the total volume of the leaching solution.

According to one or more embodiments, the lixiviant comprises an acid. Suitable acids include, but are not limited to, sulfuric acid, hydrochloric acid, nitric acid, or combinations thereof.

In some embodiments, the leaching solution further includes an oxidant. Suitable oxidants include, but are not limited to, one or more of a ferric ion, hydrogen peroxide, a nitrate anion, sodium chlorate, or combinations thereof.

In one or more embodiments, the leaching solution further includes an iron oxidizing bacterium and/or a sulfur oxidizing bacterium. Iron oxidizing bacterium includes, for example, thiobacillus ferrooxidans, acidithiobacillus ferrooxidans, or combinations thereof. Sulfur oxidizing bacterium includes, for example, sulfobacillus disulfidooxidans.

In various embodiments, the leaching solution further includes an ore comprising metal-sulfide species. The ore may be in the form of a plurality of particles, a plurality of agglomerates, a concentrate, a matte, or combinations thereof.

According to embodiments, the ore is a copper-bearing ore containing sulfide species. For example, the copper-bearing ore can include chalcopyrite (CuFeS₂), bornite (Cu₅FeS₄), enargite (Cu₃AsS₄), tetrahedrite (Cu₁₂Sb₄S₁₃), tennantite (Cu₁₂As₄S₁₃), covellite (CuS), chalcocite (Cu₂S), copper sulfide of the formula CuₓS_{y} wherein an atomic ratio of x:y is about 1 to about 2, carrolite (CuCo₂S₄), or combinations thereof. In some embodiments, the ore is a non-copper bearing ore comprising sulfide species. For example, the non-copper bearing ore can include, but is not limited to, millenite (NiS), pentlandite (Fe₉Ni₉S₁₆), molybdenite (MoS₂), violarite (FeNi₂S₄), cobaltite (CoAsS), pyrite (FeS₂), linnaeite (Co₃S₄), sphalerite ((Zn,Fe)S), cattierite (CoS₂), or combinations thereof.

In some embodiments, the leaching solution further includes metal values. Suitable metal values include, but are not limited to, at least one of copper, cobalt, nickel, zinc, molybdenum, vanadium and/or iron.

Further disclosed herein are methods of recovering metal values from an ore comprising metal sulfide species by contacting the ore with a leaching solution containing one or more additive having an organic molecule with a sp3 hybridized sulfur or a sulfoxide as described herein. According to embodiments, such methods can include leaching the ore (e.g., by in-situ leaching, heap leaching, percolation leaching, and/or agitation/tank leaching) with a leaching solution containing an organic molecule having at least one of a sp3 hybridized sulfur and/or a sulfoxide according to embodiments described herein to form a pregnant leaching solution. In some embodiments, the methods include extracting metal values from the pregnant leaching solution using a solvent extraction process, wherein the solvent extraction process forms a metal-rich organic stream. Embodiments of methods further include recovering metal values from the metal-rich organic stream, optionally wherein recovering metal values is by an electrowinning process. In some embodiments, the metal values comprise copper, and the method provides a copper recovery from chalcopyrite ore of about 95 wt % to about 99 wt %, or any individual value or sub-range within this range, in 200 days when performed at 22 degrees Celsius at atmospheric pressure; or a copper recovery from chalcopyrite ore of about 99 wt % in about 75 days, or any individual value or sub-range within this range, when performed at 22 degrees Celsius at atmospheric pressure. In some embodiments, the method further includes preparing a concentrate of the ore containing metal-sulfides using a flotation technique prior to leaching. In some embodiments, the methods further include preparing a concentrate of metal sulfide ore using a smelter process to produce a matte prior to leaching. In one or more embodiment, the matte is a nickel-copper matte or a lead-zinc matte.

### DETAILED DESCRIPTION

Reference will now be made in detail to the example embodiments of this disclosure, which are illustrated in the accompanying drawings. Wherever possible, the same reference numbers will be used throughout the drawings to refer to the same or like parts throughout the several views. Features of the various embodiments described herein may be combined with each other, unless specifically noted otherwise. The details of one or more embodiments of the invention are set forth in the description below. Other features, objects, and advantages of the invention will be apparent from the description and from the claims.

The present disclosure relates generally to the field of mining and more specifically, to leaching a target metal such as copper from one or more of the following sources of ore: (1) metal-sulfide ore, (2) ore concentrate, (3) leaching residue, (4) a matte, and/or (5) tailings produced by upstream processing of ore, for example, magnetic separation or flotation. The term “matte” as used herein refers to the product of a smelter, for example, a crude mixture of molten sulfides formed as an intermediate product of the smelting of sulfide ores of metals. The “matte” contains a metal (e.g., copper, nickel, lead) and sulfur, which can be further refined to obtain a pure metal. Instead of being smelted directly to metal, copper ores are usually smelted to matte, for example, containing 40-45 percent copper along with iron and sulfur, which is then treated by converting in a Bessemer-type converter. Air is blown into the molten matte, oxidizing the sulfur to sulfur dioxide and the iron to iron oxide that combines with a silica flux to form slag, leaving the copper in the metallic state. Smelting of nickel sulfide ores yields a matte in which nickel and copper make up about 15 percent, iron about 50 percent, and sulfur the remainder; the iron is removed in a converting furnace, and the sulfides of copper and nickel are separated before being reduced to the metals. Smelting of lead sulfide ores produces a liquid layer of copper sulfide matte that can be decanted, along with slag and speiss, from the lead bullion.

More particularly, the disclosure relates to the addition of a chemical additive comprising of an organic sp3 hybridized sulfur and/or sulfoxide to increase the rate of dissolution of a base metal from the sulfidic ore, such as copper from chalcopyrite (CuFeS₂). Redistribution of the energy of orbitals of individual atoms to give orbitals of equivalent energy happens when two atomic orbitals combine to form a hybrid orbital in a molecule. This process is called hybridization. During the process of hybridization, the atomic orbitals of comparable energies are combined together and the process typically involves the merging of an ‘s’ orbital with ‘p’ orbitals or alternatively an ‘s’ orbital with ‘d’ orbitals. The new orbitals thus formed are known as hybrid orbitals. Hybrid orbitals are useful in explaining atomic bonding properties and molecular geometry. Methane gas (CH₄) is a good example to consider for hybridization theory. The carbon atom forms four (4) single bonds wherein the valence-shell s orbital combines with three (3) valence-shell p orbitals. This combination leads to the formation of four (4) equivalent sp3 hybrid orbitals. These will have a tetrahedral arrangement around the carbon atom, which is bonded via these hybrid orbitals to four (4) different hydrogen atoms.

The term “sp3 hybridization” refers to a molecule containing one ‘s’ orbital and three (3) ‘p’ orbitals belonging to the same shell of an atom, that have combined together to form four (4) equivalent orbitals, referred to as tetrahedral hybridization. The new orbitals formed are called sp3 hybrid (or hybridized) orbitals. Each sp3 hybrid orbital has 25% s character and 75% p character. Additional examples of molecules containing sp3 hybridized orbitals include the carbon atoms in ethane (C₂H₆) and chloromethane (CH₃Cl), and both the carbon and sulfur atoms in dimethyl sulfide (CH₃SCH₃).

Base metal containing ores are typically classified into two categories-oxidic and sulfidic ores. Oxidic ores (e.g., cuprite, malachite, and azurite) are generally found near the surface as they are often the oxidation products of the typically deeper primary and secondary sulfidic ores or have formed in areas where metals have leached from the surface in aqueous solution, becoming trapped and concentrated in the host formation. Sulfidic ore deposits (e.g., chalcopyrite, bornite, and chalcocite) typically arise in either igneous rock, formed during the original cooling of the rock from the mantle, or through hydrothermal transformation of metal rich oxidic deposits formed from surface leaching. Sulfidic ores are generally sub-divided into two groups, primary and secondary. Primary sulfidic ores are generally in the initial state whereas secondary sulfidic ores have undergone some subsequent weathering and oxidation processes.

Depending on the chemical nature of metal bearing ore, mines typically process the ore to extract and concentrate the target valuable metal(s) through either a selective flotation or hydrometallurgical process. Selective flotation, most commonly used for sulfidic ores, requires the ore to be finely ground to liberate individual grains of metal containing mineral from the waste (gangue) material. The ground ore is processed as an aqueous slurry, with the addition of surface-active chemicals which facilitate the flotation and separation of the metal containing sulfide minerals from the gangue. The recovered metal sulfide minerals can then be further processed to recover the metal(s), often by pyrometallurgical processing. However, selective flotation requires the ore to be ground down to a relatively small particle size, which consumes large amounts of energy, and all of the waste (gangue) material is also in liquid (slurry) form, requiring further processing before disposal. Consequently, the hydrometallurgical process methods are especially important for recovering metal from low grade ores, where the ratio of metal values to the waste material is low, and more complex processing methods are not economically viable.

In hydrometallurgical processing, the ore is treated with a lixiviant which dissolves the target valuable metal(s) into aqueous solution, leaving the waste (gangue) material in the solid phase. The aqueous solution containing the solubilized metal(s) is collected and separated from the gangue material and the metal(s) can then be recovered in a more concentrated form by one or more processes such as precipitation and/or solvent extraction.

During hydrometallurgical processes, metal is extracted when the metal-containing material is leached in one of several ways. Leaching is typically accomplished by applying an aqueous solution containing a lixiviant to a collection of the ore and/or a concentrate. A common lixiviant is sulfuric acid (“H₂SO₄”) because it provides efficient and cost-effective liberation of the metal from the ore. The leaching process can be an in-situ, heap, dump, percolation, and/or agitation leaching process. However, for all the leaching methods, the intrinsic principles of leaching are the same, the process: 1) can dissolve the ore minerals rapidly enough to make commercial extraction possible and the lixiviant shows chemical inertness toward the gangue minerals because in situations where gangue minerals are attacked, an excessive amount of the lixiviant is consumed and the leach liquor will be fouled with impurities to an undesirable extent; 2) are cheap and readily obtainable in large quantities; and 3) can be regenerated in the subsequent processes following leaching. The underpinning characteristic of leaching is that regardless of the lixiviant used, it interacts with the ore particles in a way that allows for transfer of the desired metal from the ore into a collected and then managed solution.

Heap leaching is a common method of leaching in hydrometallurgical processes. When metal-containing material is piled into a heap and wetted with a solution of lixiviant, significant time is required for the solution to percolate down through the heap before it can be collected and supplied to subsequent operations. The extraction process can require several days to months.

The leaching of metal bearing ore typically requires the use of a solution such as an acid, a base and/or an oxidant (e.g., a ferric ion) to facilitate recovery target metals (e.g., copper) into a lixiviant. This process is commonly applied for oxide minerals of metal-bearing ores, where the target metal is subsequently recovered from the lixiviant by means of solvent extraction/electrowinning (SX/EW), direct electrowinning, cementation, and/or precipitation reactions to create a salable metal product.

Historically, many mining companies have chosen to recover metal from predominantly sulfidic ore sources such as chalcopyrite, bornite, molybdenite, and many others by pyrometallurgical processing methods that typically occur after crushing, grinding, and flotation operations in many mines throughout the world. As ore grades have begun to decline globally, there is a desire by many mining companies to process lower grade sulfidic ores via a hydrometallurgical technique. However, leaching rates of sulfidic ores in standard leaching solutions such as dilute sulfuric acid tend to be quite slow with low recovery rates observed from predominant target minerals such as chalcopyrite, where only 20-40% of the total available copper may be recovered under nominal leaching conditions. This has been shown to be the result of the formation of an iron depleted, copper rich, covellite like layer on the surface of the chalcopyrite which is resistant to chemical oxidation and acts as a passivating film. Typically, only 20-25% of total copper may be recovered from chalcopyrite by conventional leaching techniques before passivation prevents further copper recovery.

Disclosed herein according to various embodiments are a group of chemical additives that can be added to the lixiviant in the leaching process to (a) increase the rate of dissolution of metal sulfide ores and/or mattes and (b) enhance the oxidation and dissolution of metal sulfide ores past the point of passivation commonly seen under standard leaching conditions. The term “passivation” as used herein refers to oxidizing or protecting the sulfide surface of an ore from water and oxygen. The additives described were selected in part due to their chemistry being compatible with downstream processes such as SX/EW or direct electrowinning.

Compositions, methods of preparation and methods of use thereof according to embodiments herein can improve the leaching efficiency of a target metal during an ore leaching process. The leaching of some ore minerals (e.g., primary and secondary sulfide minerals) can be oxygen dependent. For ores that are leachable through oxidation, diffusion of the oxidant to the surface of the mineral ore can increase the leaching efficiency. Notably, copper sulfide ores such as chalcopyrite are the most abundant naturally occurring copper mineral, estimated to account for about 70% of the copper deposits found in the earth's crust, but are difficult to leach with conventional methods (e.g., using sulfuric acid).

Compositions and methods described herein increase the recovery of target metals (e.g. copper) from ores (e.g., primary and/or secondary sulfide ores) through the utilization of an additive having at least one organic molecule with a sp3 hybridized sulfur and/or sulfoxide in combination with a lixiviant. This combination increases the leaching efficiency of the ore at ambient temperature. In some embodiments, the target metal values include copper, and methods as described herein provide a copper recovery from chalcopyrite of about 50 wt % to about 100 wt %, about 75 wt % to about 99.9 wt %, about 90 wt % to about 99 wt %, or any individual value or sub-range therein, under standard temperature and pressure conditions (e.g., 20-25 °C, 1.0 atm).

Compositions and methods as described herein include a chemical additive (e.g., a leaching aid) to a lixiviant (e.g., an acid) used to recover a target metal (e.g., copper) from a mined ore containing metal-sulfide species (e.g., chalcopyrite, that is, CuFeS₂). In various embodiments, the chemical additive includes at least one organic sp3 hybridized sulfur atom and/or a sulfoxide group, and a wide variety of such molecules have been identified as being suitable in the disclosed compositions and methods. Examples of suitable chemical additives include, but are not limited to, cystine, cysteine, cysteamine, dimethyl-sulfoxide, methionine, or combinations thereof.

Chemical additives according to embodiments herein can enhance and increase the rate of metal recovery from sulfide ores. This is of great advantage to the mine as it helps increase the yields from existing heaps and leach residue and avoids processes such as flotation and/or smelting to recover valuable metals from sulfide ores.

According to various embodiments, the addition of a chemical additive containing at least one organic molecule having a sp3 hybridized sulfur and/or sulfoxide in small concentrations to a hydrometallurgical leaching process can increase the rate of dissolution of target metals from sulfidic ores, concentrates, tailings, and leach residues, and enhance the oxidation and total dissolution that is achieved relative to similar leaching conditions conducted in the absence of the chemical additive.

Compositions and methods described herein relate to the leaching of metal-sulfide containing ores, ore concentrate, leaching residue, or tailings produced by upstream processing of ore such as magnetic separation or flotation. According to various embodiments, methods described herein relate to recovery of a least one target metal from an ore, ore concentrate, leach residue, matte, and/or mine tailings containing at least one metal sulfide, such as, but not limited to, chalcopyrite. This recovery can be completed in acidic conditions with a solution pH of <4.0.

### Leaching Solutions

The leaching of primary sulfide minerals such as chalcopyrite (CuFeS₂) has been well studied and documented in literature. There are two main equations that govern the leaching of chalcopyrite in ferric rich systems:

CuFeS₂+4Fe³⁺→Cu²⁺+5Fe²⁺+2S⁰ 1.

CuFeS₂+4Fe³⁺+3O₂+2H₂O→Cu²⁺+5Fe²⁺+2H₂SO₄ 2.

Currently about 75% of chalcopyrite leaching occurs via Reaction 1. Increasing the availability of oxygen helps more chalcopyrite to leach via Reaction 2, thereby increasing the kinetic rate of copper recovery. Also essential to this leaching process is the regeneration of ferric ion, which occurs via Reaction 3.

4Fe²⁺+4H⁺O₂→4Fe³⁺+2H₂O 3.

Without being bound by any particular theory, it is believed that the mechanism of action relates to the use of a sp3 hybridized sulfur (i.e., the orbital structure around an atom of sulfur in a molecule) and/or to a sulfoxide such that the resulting molecule(s) is(are) designed to be soluble in raffinate. Improved molecules also feature another functional group that can function as a Lewis base to facilitate the coordination of a metal in the sulfide matrix such as an imine, carbonyl, amine, pyridine, azole, etc. It is believed that this increase in electron density around the cuprous (Cu⁺) atom in chalcopyrite facilitates the extraction of the copper from the mineral matrix and its oxidation to cupric (Cu²⁺) and increases the dissolution rate of the chalcopyrite.

Disclosed herein are various embodiments of a leaching solution that includes a leaching additive containing an organic compound having a sp3 hybridized sulfur and/or in combination with a lixiviant. In some embodiments, the leaching additive comprises one or more compound of Formula (A) or Formula (B):

R¹—S—R² (A)

R¹—(S═O)—R² (B)

wherein R¹ and R² is each, independently, selected from the below groups:
H, with the proviso that when either R^{t} or R² is H, then R¹ and R² are not the same;
C₁ to C₁₀ linear or branched alkyl group which may be interrupted by one or more functional groups;
C₁ to C₁₀ hydroxyalkyl group, a ether thereof or an ester thereof;
C₁ to C₁₀ aminoalkyl group or a nitrogen functionalized derivative thereof,
C₁ to C₁₀ carboxyalkyl;
an amino acid residue having the structure —(CₙH₂ₙ)CH(NH₂)COOH where n is an integer between 0 and 6—this structure may have straight and/or branched alkyl groups;
C₂ to C₁₀ alkenyl group;
C₂ to C₁₀ alkynyl group;
a carbonyl group having the structure —C(═O)R³;
C₁ to C₁₀ alkoxy group;
C₂ to C₁₀ alkenyloxy group;
C₂ to C₁₀ alkynyloxy group;
C₂ to C₁₀ alkenylamino group;
C₂ to C₁₀ dialkenylamino; or
a sulfide group having the formula S—R⁴,
wherein R³ and R⁴ is each, independently, H, OH, NH₂, a C₁ to C₁₀ linear or branched alkyl group which may be interrupted by one or more functional groups, C₁ to C₁₀ hydroxyalkyl group, a ether thereof or an ester thereof, C₁ to C₁₀ aminoalkyl group or a nitrogen functionalized derivative thereof, C₁ to C₁₀ carboxyalkyl, an amino acid residue, C₂ to C₁₀ alkenyl group, C₂ to C₁₀ alkynyl group, carbonyl group, C₁ to C₁₀ alkoxy group, C₂ to C₁₀ alkenyloxy group, C₂ to C₁₀ alkynyloxy group, C₂ to C₁₀ alkenylamino group, C₂ to C₁₀ dialkenylamino, or a sulfide group,
optionally, wherein Formula (A) or Formula (B) is cyclic.

Examples of suitable organic additives are provided below:

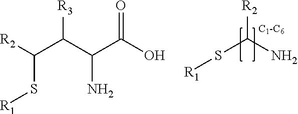

wherein R¹ to R³ is each, independently, selected from the groups defined for R¹ and R² above with respect to Formulas (A) and (B).

In some embodiments, only one of R^{t} or R² is H. In certain embodiments, only one or only two of R¹, R², R³ or R⁴ is H. In some embodiments, when R¹ is H, R² is not H or when R² is H, R¹ is not H. In some embodiments, Formulas (A) and (B) meet the proviso that when either R¹ and R² is H, then R¹ and R² are not the same.

In various embodiments, the linear or branched C₁ to C₁₀ alkyl group can include or be interrupted with one or more functional group (e.g., ethers, esters, amides, carboxyls). In some embodiments, when R¹ is methyl, R² is not H or methyl or when R² is methyl, R² is not H or methyl. In some embodiments, when R¹ is methyl, R² is a C₄ alkyl optionally substituted with one or more amine and/or carboxyl group.

According to various embodiments, the C₁ to C₁₀ hydroxyalkyl group can include, but is not limited to, methylol and/or 2-hydroxyethyl or ethers and/or esters thereof. In some embodiments, the C₁ to C₁₀ aminoalkyl group can include, but is not limited to, aminomethyl and/or 2-aminoethyl and optionally can include nitrogen functionalized derivatives thereof such as N-alkylaminoalkyl.

According to embodiments, the C₁ to C₁₀ carboxyalkyl group can include, but is not limited to, carboxymethyl and/or 2-carboxyethyl or common derivatives such as esters and amides thereof.

In some embodiments, the amino acid residue can include any chemical method of adjoining an alpha- (α-), beta- (β-), gamma- (γ-) or delta- (δ-) amino acid.

According to embodiments, the C₁ to C₁₀ hydroxyalkyl group can include, but is not limited to methylol and/or 2-hydroxyethyl. In some embodiments, the sulfide groups S—R⁴, where R⁴ is defined as R¹ or R² set forth above can include, but is not limited to, the alkyl groups, alkenyl groups, alkynyl groups and/or carbonyl groups.

In some embodiments, the leaching solution and/or leaching additive does not comprise one or more of thiocarbonyl functionality or group, sulfonic acid functionality or group and/or an alkyl bridged polyamine. In embodiments, the leaching solution and/or leaching additive is free of one or more of thiourea, ethylene thiourea, formamidine disulfide, a sulfonamide, methane sulfonic acid, ethylenediamine, polyethylenimine, imidazole, diethylenetriamine, triethylenetetramine, tetraethylenepentamine, 1,2-diaminopropane, 2,3-butanediamine, or combinations thereof.

According to various embodiments, the leaching additive is a sp3 hybridized sulfur and/or sulfoxide containing organic molecule. The leaching additive may be present in an amount of about 0.001 mM to about 67 mM, 0.1 mM to about 2 mM, or any individual value or sub-range within these ranges, based on the total volume of the leaching solution. In some embodiments, the leaching solution includes metal values. Suitable metal values include, but are not limited to, at least one of copper, cobalt, nickel, zinc, molybdenum, vanadium, iron, or combinations thereof.

Leaching solutions according to embodiments herein may have a pH of less than about 4, less than about 3, less than about 2, less than about 1, about 0.1 to about 4.0, about 1 to about 2.5, or any individual value or sub-range within these ranges. The lixiviant can comprise an acid, such as, sulfuric acid, nitric acid, or combinations thereof. In some embodiments, the leaching solution further includes an oxidant. Suitable oxidants include, but are not limited to, a ferric ion, hydrogen peroxide, a nitrate anion, sodium chlorate, or combinations thereof.

According to various embodiments, the leaching solution may include at least one of an iron oxidizing bacterium (e.g., thiobacillus ferrooxidans, acidithiobacillus ferrooxidans) or a sulfur oxidizing bacterium (e.g., sulfobacillus disulfidooxidans). One or more iron oxidizing bacterium may be included to convert ferrous ions to ferric ion during the leaching process. One or more sulfur oxidizing bacterium may be included to convert elemental sulfur produced during leaching to sulfate in the generation of acid. In some embodiments, these bacterium are present on the surface of the ore as it resides in its natural environment. The leaching solution may contain ore-derived bacteria as a result at a concentration of less than about 1000 ppm. In other embodiments, the bacterium from an external source may be added to the leaching solution, for example, at a concentration of greater than 1000 ppm. Bacterium from an external source may be added to a leaching solution to inoculate various species and assist in the leaching process.

According to various embodiments, the leaching solution further includes a metal sulfide ore. The metal sulfide ore may be in the form of at least one of a plurality of particles, a plurality of agglomerates, a concentrate, a matte, or combinations thereof. In some embodiments, the metal sulfide ore is a copper-bearing sulfide ore. Suitable copper-bearing sulfide ore includes, but is not limited to, chalcopyrite (CuFeS₂), bornite (Cu₅FeS₄), enargite (Cu₃AsS₄), tetrahedrite (Cu₁₂Sb₄S₁₃), tennantite (Cu₁₂As₄S₁₃), covellite (CuS), chalcocite (Cu₂S), carrolite (CuCo₂S₄), or combinations thereof. In some embodiments, the metal sulfide ore is a non-copper bearing sulfide ore. Suitable non-copper bearing sulfide ore includes, but is not limited to, one or more of millenite (NiS), pentlandite (Fe₉Ni₉S₁₆), molybdenite (MoS₂), violarite (FeNi₂S₄), cobaltite (CoAsS), pyrite (FeS₂), linnaeite (Co₃S₄), sphalerite ((Zn,Fe)S), cattierite (CoS₂), or combinations thereof.

### Methods of Preparation

Further described herein are methods of preparing additives and leaching solutions according to various embodiments. In some embodiments, the methods include combining one or more leaching additives (e.g., comprising a sp3 hybridized sulfur and/or a sulfoxide) with a lixiviant to form the leaching solution. The one or more leaching additives can be added at a concentration of about 0.001 mM to about 67 mM, 0.1 mM to about 2 mM, or any individual value or sub-range within these ranges, based on the total volume of the leaching solution.

Methods according to embodiments herein can include adding one or more leaching additives as described herein to a leach liquor from a mining process. The method may further include adjusting the concentration of the one or more leaching additives in the leach liquor to maintain a concentration of up to about 10 g/L, up to about 5 g/L, up to about 2.5 g/L, up to about 1.5 g/L, up to about 0.5 g/L, or up to about 0.01 g/L, or any individual value or sub-range within these ranges, in the leach liquor at about 0.05 mM to about 70 mM, about 0.5 mM to 1 mM, or any individual value or sub-range within these ranges.

In some embodiments, the methods include adjusting the pH of the leaching solution to less than about 4, less than about 3, less than about 2, less than about 1, about 0.1 to about 4.0, about 1 to about 2.5, or any individual value or sub-range within these ranges. In some embodiments, adjusting includes adding lixiviant in a greater amount and/or at various concentrations. The lixiviant can comprise an acid, such as, sulfuric acid, nitric acid, or combinations thereof.

In some embodiments, the method includes adding an oxidant to the leaching solution. Suitable oxidants include, but are not limited to, one or more of a ferric ion, hydrogen peroxide, a nitrate anion, sodium chlorate, or combinations thereof.

According to various embodiments, the methods can include adding (e.g., from an external source) an iron oxidizing bacterium (e.g., thiobacillus ferrooxidans, acidithiobacillus ferrooxidans) and/or a sulfur oxidizing bacterium (e.g., sulfobacillus disulfidooxidans) leach liquor or leaching solution. One or more iron oxidizing bacterium may be added to convert ferrous ions to ferric ion during the leaching process. One or more sulfur oxidizing bacterium may be added to convert elemental sulfur produced during leaching to sulfate in the generation of acid. In some embodiments, an inoculation of the various species listed may be added to assist in the leaching process.

According to various embodiments, the method includes combining a metal sulfide ore with the leaching solution. The metal sulfide ore may be in the form of a plurality of particles, a plurality of agglomerates, a concentrate, a matte, or combinations thereof. In some embodiments, the metal sulfide ore is a copper-bearing sulfide ore. Suitable copper-bearing sulfide ore includes, but is not limited to, chalcopyrite (CuFeS₂), bornite (Cu₅FeS₄), enargite (Cu₃AsS₄), tetrahedrite (Cu₁₂Sb₄S₁₃), tennantite (Cu₁₂As₄S₁₃), covellite (CuS), chalcocite (Cu₂S), carrolite (CuCo₂S₄), or combinations thereof. In some embodiments, the metal sulfide ore is a non-copper bearing sulfide ore. Suitable non-copper bearing sulfide ore includes, but is not limited to, millenite (NiS), pentlandite (Fe₉Ni₉S₁₆), molybdenite (MoS₂), violarite (FeNi₂S₄), cobaltite (CoAsS), pyrite (FeS₂), linnaeite (Co₃S₄), sphalerite ((Zn,Fe)S), cattierite (CoS₂), or combinations thereof.

### Methods of Use

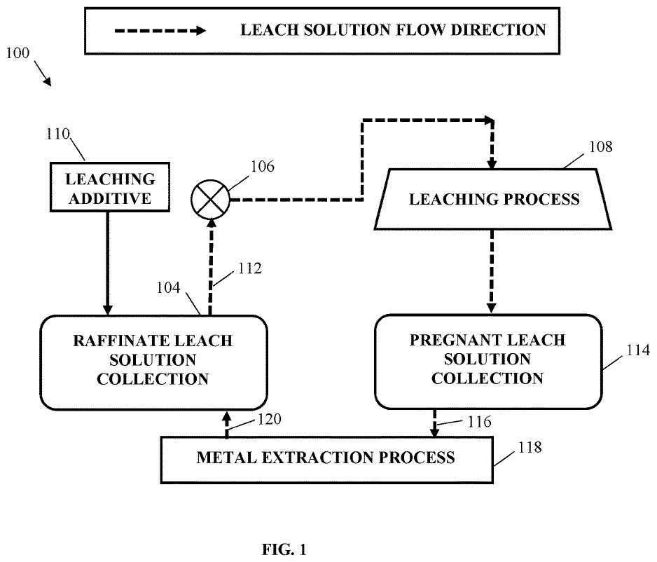

Further disclosed herein are methods ofusing leaching solutions according to embodiments to extract metal values from ore. Such methods can include contacting the leaching solution with an ore to form a metal-rich leaching solution. FIG. 1 shows a schematic diagram of an embodiment of a leaching system 100 suitable for forming and using compositions according to embodiments herein to leach a metal-containing material.

In some embodiments, disclosed are methods for increasing the recovery of target metals from ore (e.g., primary sulfide ores). The method can include contacting the ore with a leaching solution comprising one or more leaching additives according to embodiments herein and extracting one or more target metals at a higher concentration than in a solution that does not contain the described leaching additives.

The methods described herein can be applied to leaching applications such as, but not limited to, in-situ, heap/percolation leaching, and/or vat/tank leaching. The ore may contain a variety of particle size fragments. In some cases, the ore may be agglomerated prior to beginning the leaching process. In other embodiments, a concentrate of metal-sulfide ore may be prepared by flotation techniques prior to leaching. In other embodiments, a concentrate of metal sulfide ore may be prepared as a result of a smelter process to produce a matte, such as a nickel-copper or lead-zinc matte prior to leaching.

In embodiments, methods of recovering metal values from a metal sulfide ore, include leaching the metal sulfide ore with a leaching solution containing an organic molecule with a sp3 hybridized sulfur and/or sulfoxide according to form a pregnant leaching solution. The methods further include extracting metal values from the pregnant leaching solution using a solvent extraction process, wherein the solvent extraction process forms a metal-rich organic stream; and recovering metal values from the metal-rich organic stream (e.g., using an electrowinning process).

Metal values can include copper, cobalt, nickel, zinc, molybdenum, vanadium, iron, or combinations thereof. In some embodiments, the lixiviant includes sulfuric acid. In one or more embodiments, the metal values include copper, wherein the method provides an improved copper recovery from chalcopyrite ore at atmospheric temperature and pressure as compared to a method of leaching ore with a leaching solution that does not contain organic additives according to embodiments herein.

### Examples

To test the efficacy of the chemical additives described above, the following experimental procedures were conducted using samples of naturally occurring minerals.

### Test Procedure 1

“Agitation Testing” or “stirred jar test” experiments in the presence of leaching additives according to embodiments herein and a comparative additive were conducted in triplicate. All volumes and masses were measured to within 1% of the indicated amount. Mineral samples were crushed and sieved down to less than the required size for the experiment. The respective leaching additives according to embodiments herein and where tested, the comparative additive was dosed into the lixiviant solution at the required concentration for the respective tests. Each translucent “wide mouth” 500 mL polypropylene bottle was charged with the required amount of mineral and 400 mL of lixiviant solution containing the respective additive at the listed concentration. The fill line was marked on each bottle using a permanent marker. Each bottle was fitted with a standard 1.5-inch polypropylene disk impeller and lid with a central 1 cm hole which leaves an air gap between the impeller shaft and the lid.

The samples were placed into a water bath maintained at the desired temperature and the impellers were fitted to a digital over-head mixer. The impeller was elevated 5 cm from the bottom of each bottle. Stirring was commenced at 1000 rpm. The fill level of each bottle was maintained using deionized water to account for evaporative losses. Samples were collected when required by turning off the impeller and allowing the solution to settle for 10 min. A volume of 2.5 mL of the solution were drawn from 1 cm below the surface using a disposable plastic pipette and filtered through a Whatman 4 filter paper. Samples were analyzed using ICP-OES for the target metal content. The percent of total metal (e.g., copper) recovered from the mineral was calculated and the results were presented as a percentage verses the control (i.e., no leaching additive). Percent improvement in metal recovery was calculated using the following formula:

$$Δ Metal Recovery = \frac{Additive Result - Control Result}{Control Result} \times 100 \%$$

### Leaching Additives Tested

TABLE 1
List of Chemical Additives Screened

| Chemical | Product |
|---|---|
| (2-Imino-4-Oxo-Thiazolidin-5-YI)-Acetic Acid | Additive 1 |
| Methionine | Additive 2 |
| Methionine Sulfoxide | Additive 3 |
| (2-4-Oxo-Thiazolidin-5-YI)-Acetic Acid | Additive 4 |
| Cystine | Additive 5 |
| Glycine | Additive 6 |
| Dimethyl-Sulfoxide | Additive 7 |
| Thiodiglycol | Additive 8 |
| 2,2′-Thiobis(ethylamine) | Additive 9 |
| Benzotriazole | Additive 10 |
| Cysteine | Additive 11 |

### Example 1—Agitation Testing to Extract Copper from Chalcopyrite Mineral

A number of additives were tested according to Test Procedure 1 using the following conditions:
Mineral Type: 2 g<45 μm sieved Chalcopyrite sourced from Mineral Zone with 28.5% Cu
Lixiviant: Raffinate sourced from a mine in Arizona containing 0.13 g/L Cu, 0.6 g/L Fe³+, 0.02 g/L Fe²⁺, 2.29 g/L H₂SO₄, pH 1.82, ORP=644 mV (1M Ag/AgCl)
Leaching Additive Dose: The additives tested were all dosed at 0.67 mM.
Test Temperature: All tests were conducted at 25 °C in a water bath.
Results reported after 30 days of agitation.

TABLE 2
Agitation Testing to extract Copper from Chalcopyrite Mineral

| | Copper | Delta Copper Recovery |
|---|---|---|
| Product | Recovered (%) | Vs. Control (%) |
| Control | 14.7 | — |
| Additive 1 | 12.5 | −15 |
| Additive 2 | 38.7 | 164 |
| Additive 3 | 6.4 | −57 |
| Additive 5 | 16.8 | 15 |
| Additive 6 | 13.5 | −8 |
| Additive 7 | 14.5 | −1 |
| Additive 8 | 15.4 | 5 |
| Additive 9 | 11.1 | −25 |
| Additive 10 | 12.0 | −18 |
| Additive 11 | 16.0 | 9 |

From the testing results displayed in Table 2, Additives 2, 5, and 11 were found to increase the total recovered amount of copper significantly relative to the control.

### Example 2—Agitation Testing to Extract Copper from Chalcopyrite Mineral

Three additives were tested according to Test Procedure 1 using the following conditions:
Mineral Type: 1 g<45 μm sieved Chalcopyrite sourced from Rocks, Gems, and Minerals with 19.1% Cu purity.
Lixiviant: Synthetic Raffinate Prepared containing 2.5 g/L Fe³⁺, 0.6 g/L Fe²⁺, 0.1 g/L H₂SO₄, pH 2.0, ORP=506 mV (1M Ag/AgCl)
Leaching Additive Dose: The additives tested were all dosed at 100 mg/L.
Test Temperature: All tests were conducted at 23 °C in a water bath.
Results reported after 69 days of agitation.

TABLE 3
Agitation Testing to extract Copper from Chalcopyrite Mineral

| | Copper | Delta Copper Recovery |
|---|---|---|
| Product | Recovered (%) | Vs. Control (%) |
| Control | 37.0 | 0 |
| Additive 1 | 31.4 | −15 |
| Additive 2 | 94.9 | 156 |
| Additive 4 | 39.3 | 6 |

From the testing results displayed in Table 3, Additives 2 and 4 were found to increase the total recovered amount of copper significantly relative to the control.

### Example 3—Agitation Testing to Extract Copper from Chalcopyrite Mineral

Three additives were tested according to Test Procedure 1 using the following conditions:
Mineral Type: 2 g<45 μm sieved Chalcopyrite sourced from Mineral Zone with 28.5% Cu purity.
Lixiviant: Synthetic Raffinate Prepared containing 2.5 g/L Fe³⁺, 0.6 g/L Fe²⁺, 0.1 g/L H₂SO₄, pH 2.0, ORP=506 mV (1M Ag/AgCI)
Leaching Additive Dose: The additives tested were all dosed at 100 mg/L.
Test Temperature: All tests were conducted at 23 °C in a water bath.
Results reported after 49 days of agitation.

TABLE 4
Agitation Testing to extract Copper from Chalcopyrite Mineral

| | Copper | Delta Copper Recovery |
|---|---|---|
| Product | Recovered (%) | Vs. Control (%) |
| Control | 16.6 | 0 |
| Additive 2 | 33.8 | 103 |
| Additive 3 | 13.1 | −21 |

From the testing results displayed in Table 4, Additive 2 was the only product screened that was found to increase the total recovered amount of copper significantly relative to the control.

### Example 4—Agitation Testing to Extract Copper from Chalcopyrite Mineral

Additive 2 was tested at a range of dosages according to Test Procedure 1 using the following conditions:
Mineral Type: 2 g<45 μm sieved Chalcopyrite sourced from Mineral Zone with 28.5% Cu
Lixiviant: Raffinate sourced from a mine in Arizona containing 0.13 g/L Cu, 0.6 g/L Fe³⁺, 0.02 g/L Fe²⁺, 2.29 g/L H₂SO₄, pH 1.82, ORP=644 mV (1M Ag/AgCl)
Leaching Additive Dose: Varied from 0 to 1000 mg/L
Test Temperature: All tests were conducted at 25 °C in a water bath.
Results reported after 44 days of agitation.

TABLE 5
Agitation Testing to extract Copper from Chalcopyrite Mineral

| Additive 2 | Copper | Delta Copper Recovery |
|---|---|---|
| Dose (mg/L) | Recovered (%) | Vs. Control (%) |
| 0 | 8.0 | 0 |
| 25 | 14.0 | 74 |
| 50 | 13.5 | 68 |
| 100 | 15.6 | 94 |
| 200 | 22.0 | 174 |
| 500 | 34.1 | 324 |
| 1000 | 35.0 | 335 |

From the testing results displayed in Table 5, the total copper recovery increased with increasing dose of Additive 2.

### Example 5—Agitation Testing to Extract Metal Front Different Mineral Species

Additive 2 was tested according to Test Procedure 1 using the following conditions:
Mineral Types: 2 g<45 μm sieved covellite sourced from Sigma Aldrich with 66.5% Cu, 2 g<45 μm sieved Copper Concentrate (Cu Con) sourced from a mine with 32% Cu, 2 g<45 μm sieved Ni₃S₂ sourced from Sigma Aldrich with 73.3% Ni
Lixiviant: Raffinate sourced from a mine in Arizona containing 0.13 g/L Cu, 0.6 g/L Fe³⁺, 0.02 g/L Fe²⁺, 2.29 g/L H₂SO₄, pH 1.82, ORP=644 mV (1M Ag/AgCl)
Leaching Additive Dose: The additive tested was dosed at 100 mg/L.
Test Temperature: All tests were conducted at 25 °C in a water bath.
Results reported after 47 days, Ni; 28 days, covellite; and 28 days Copper Con, of agitation.

TABLE 6
Agitation Testing to extract Metal from Target Mineral

| | | | | Delta Metal Recovery Vs. | | |
|---|---|---|---|---|---|---|
| | Metal Recovered (%) | | | Control (%) | | |
| Product | Covellite | Cu Con. | Ni | Covellite | Cu Con. | Ni |
| Control | 9.2 | 26.5 | 51.2 | 0 | 0 | 0 |
| Additive 2 | 10.6 | 29.1 | 53.4 | 15 | 10 | 4 |

From the testing results displayed in Table 6, Additive 2, was found to increase the total recovered amount of copper from the copper concentrate and covellite mineral, as well as the total nickel recovery relative to a control test.

### Example 6—Agitation Testing to Extract Copper from Chalcopyrite in the Presence of 35 g/L Chloride in Addition to Additive 2

Additive 2 was tested according to Test Procedure 1 using the following conditions:
Mineral Type: 2 g<45 μm sieved Chalcopyrite sourced from Mineral Zone with 28.5% Cu
Lixiviant: Raffinate sourced from a mine in Arizona containing 0.03 g/L Cu, 0.6 g/L Fe³⁺, 0.02 g/L Fe²⁺, 2.4 g/L H₂SO₄, pH 1.67, 35 g/L Cl (as NaCl), ORP=655 mV (1M Ag/AgCl)
Leaching Additive Dose: The additive tested was dosed at 100 mg/L.
Test Temperature: All tests were conducted at 25 °C in a water bath.
Results reported after 28 days of agitation.

TABLE 7
Agitation Testing to extract Copper
from Chalcopyrite Mineral with Chloride

| | Copper | Delta Copper Recovery |
|---|---|---|
| Product | Recovered (%) | Vs. Control (%) |
| Control | 6.1 | 0 |
| Additive 2 | 13.8 | 126 |

From the testing results displayed in Table 7, Additive 2, was found to increase the total recovered amount of copper from chalcopyrite in the presence of 35 g/L chloride significantly relative to the control.

### Example 7—Agitation Testing to Extract Copper from Chalcopyrite in the Presence of 3.18 g/L Sodium Chlorate (oxidant) in Addition to Additive 2

Additive 2 was tested according to Test Procedure 1 using the following conditions:
Mineral Type: 2 g<45 μm sieved Chalcopyrite sourced from Mineral Zone with 28.5% Cu
Lixiviant: Raffinate sourced from a mine in Arizona containing 0.03 g/L Cu, 0.6 g/L Fe³⁺, 0.02 g/L Fe²⁺, 2.29 g/L H₂SO₄, pH 1.82, 3.18 g/L NaOCl₃, ORP=650 mV (1M Ag/AgCl)
Leaching Additive Dose: The additive tested was dosed at 100 mg/L.
Test Temperature: All tests were conducted at 25 °C in a water bath.
Results reported after 28 days of agitation.

TABLE 8
Agitation Testing to extract Copper
from Chalcopyrite Mineral with Chlorate

| | Copper | Delta Copper Recovery |
|---|---|---|
| Product | Recovered (%) | Vs. Control (%) |
| Control | 37.4 | 0 |
| Additive 2 | 63.0 | 69 |

From the testing results displayed in Table 7, Additive 2, was found to increase the total recovered amount of copper from chalcopyrite in the presence of sodium chlorate oxidizing agent significantly relative to the control.

### Example 8—Column Leaching Investigation Utilizing a Composition According to Embodiments Herein

A raffinate leach solution with the following characteristics: 2.66 g/L Fe, 0.85 g/L H₂SO₄, ORP of 506 mV (1M Ag/AgCl) and a pH of 1.79 was used to leach a sulfide rich ore in column testing in a laboratory. For the testing, the ore used was from a copper mill flotation feed and had a total copper grade of 1.2%, with 96.2% of the copper present as sulfide minerals, (of which 92.5% was chalcopyrite) and 3.8% as acid soluble minerals. The ore was all <¾″, with 10.8% wt in the ½″<x<¾″ fraction, 32.7% wt in the ¼″<x<½″ fraction, 28.7% wt in the Tyler 10 Mesh<x<¼″, and 27.7% wt in the 0<x<Tyler 10 Mesh Fraction.

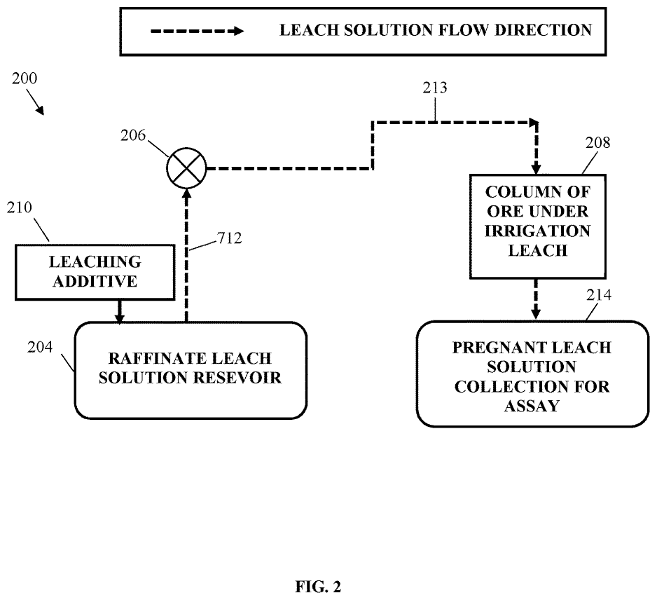

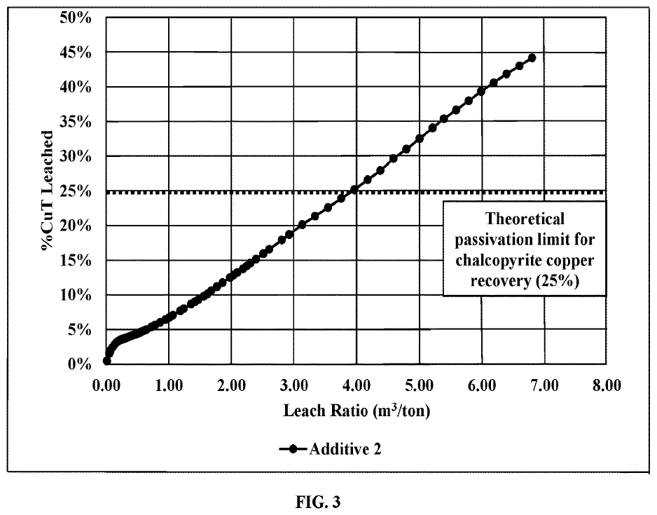

The irrigation rate was set at 1 L/hr/m² and the ore was agglomerated with 26.8 kg/ton sulfuric acid prior to column loading, which equated to 100% acid consumption of the ore. An weight of 4 kg of ore were used per column, and the test was conducted in duplicate using the setup described in FIG. 2 and the results averaged. For each sample, the mass, density, pH, and metallurgical data of the PLS were collected and reviewed. The results of this testing are shown in FIG. 3. In this testing, the same raffinate was used for each test. The columns were irrigated with the same raffinate described above that had been dosed with 100 mg/L of Additive 2 prior to irrigation starting. The columns were irrigated with a raffinate feed pumped from a jerry can that was open to the atmosphere. FIG. 3 shows that with the presence of Additive 2 in the lixiviant, copper extraction from the primary sulfide rich ore has continued on a linear trajectory over the period of testing (about 9 months) and has far exceeded the expected theoretical passivation limit of chalcopyrite of about 25%. This demonstrates that the additive is effective in promoting the oxidation of the copper beyond what would be expected under standard sulfuric acid leaching conditions at room temperature.

Reference throughout this specification to, for example, “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “an iron oxidizing bacterium” includes a single bacterium as well as a plurality of bacteria.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight. Weight percent (wt. %), if not otherwise indicated, is based on an entire composition free of any volatiles, that is, based on dry solids content.

As used herein, the term “combinations thereof” refers to an operable mixture or grouping of any two or more elements preceding the term. For example, “A, B, C, or combinations thereof” refers to “A and B,” “B and C,” “A and C” and “A, B and C.”

The foregoing description discloses example embodiments of the disclosure. Modifications of the above-disclosed assemblies, apparatus, and methods which fall within the scope of the disclosure will be readily apparent to those of ordinary skill in the art. Accordingly, while the present disclosure has been disclosed in connection with example embodiments, it should be understood that other embodiments may fall within the scope of the disclosure, as defined by the following claims.

1. A leaching solution comprising:
a leaching additive comprising an organic compound comprising a sp³ hybridized sulfur,
a sulfoxide, or a combination thereof; and
a lixiviant.

2. The leaching solution of claim 1, wherein the leaching additive comprises one or more compound of Formula (A) or Formula (B):
R¹—S—R² (A)
R¹—(S═O)—R² (B)
wherein R¹ and R² are each, independently, chosen from the following groups:
H, with the proviso that when either R¹ or R² is H, then R¹ and R² are not the same;
C1 to C10 linear or branched alkyl group which may be interrupted by one or more functional groups;
C1 to C10 hydroxyalkyl group, ana ether thereof or an ester thereof;
C1 to C10 aminoalkyl group or a nitrogen functionalized derivative thereof; C1 to C10 carboxyalkyl;
an amino acid residue having the structure —(CnH2n)CH(NH2)COOH where n is an integer between 0 and 6;
C2 to C10 alkenyl group; C2 to C10 alkynyl group;
a carbonyl group having the structure —C(═O)R³; C1 to C10 alkoxy group; C2 to C10 alkenyloxy group; C2 to C10 alkynyloxy group; C2 to C10 alkenylamino group;
C2 to C10 dialkenylamino; or
a sulfide group having the formula —S—R⁴,
wherein R³ and R⁴ is each, independently, H, OH, NH2, C1 to C10 linear or branched alkyl group which may be interrupted by one or more functional groups, C1 to C10 hydroxyalkyl group, a ether thereof or an ester thereof, C1 to C10 aminoalkyl group or a nitrogen functionalized derivative thereof, C1 to C10 carboxyalkyl, an amino acid residue, C2 to C10 alkenyl group, C2 to C10 alkynyl group, a carbonyl group, C1 to C10 alkoxy group, C2 to C10 alkenyloxy group, C2 to C10 alkynyloxy group, C2 to C10 alkenylamino group, C2 to C10 dialkenylamino, or a sulfide group,
optionally, wherein Formula (A) or Formula (B) is cyclic.

3. The leaching solution of claim 1, wherein the leaching additive comprises cystine, cysteine, cysteamine, dimethyl-sulfoxide, methionine, or combinations thereof.

4. The leaching solution of claim 1, wherein the leaching solution does not comprise an organic molecule comprising thiocarbonyl functionality, a thiocarbonyl group, sulfonic acid functionality, a sulfonic acid group, an alkyl bridged polyamine, or combinations thereof; or the leaching solution is free of thiourea, ethylene thiourea, formamidine disulfide, a sulfonamide, methane sulfonic acid, ethylenediamine, polyethylenimine, imidazole, diethylenetriamine, triethylenetetramine, tetraethylenepentamine, 1,2-diaminopropane, 2,3-butanediamine, or combinations thereof; or
the leaching solution is free of thiourea, ethylene thiourea, formamidine disulfide, ethylenediamine, polyethylenimine, imidazole, or combinations thereof.

5. (canceled)

6. The leaching solution of claim 1, comprising a pH of less than about 4.

7. The leaching solution of claim 1 wherein the lixiviant comprises an acid.

8. The leaching solution of claim 7, wherein the acid comprises sulfuric acid, hydrochloric acid, nitric acid, or combinations thereof.

9. The leaching solution of claim 1, further comprising an oxidant, or further comprising an iron oxidizing bacterium, a sulfur oxidizing bacterium, or a combination thereof, or further comprising metal values.

10. The leaching solution of claim 9, wherein the oxidant comprises a ferric ion, hydrogen peroxide, a nitrate anion, sodium chlorate, or combinations thereof.

11. (canceled)

12. The leaching solution of claim 914, wherein the iron oxidizing bacterium comprises thiobacillus ferrooxidans or acidithiobacillus ferrooxidans, and the sulfur oxidizing bacterium comprises sulfobacillus disulfidooxidans.

13. The leaching solution of claim 1, further comprising an ore comprising metal-sulfide species.

14. The leaching solution of claim 13, wherein the ore is in the form of a plurality of particles, a plurality of agglomerates, a concentrate, a matte, or combinations thereof.

15. The leaching solution of claim 14, wherein the ore is a copper-bearing ore containing sulfide species or the ore is a non-copper bearing ore comprising sulfide species.

16. The leaching solution of claim 15, wherein the copper-bearing ore comprises at least one of chalcopyrite (CuFeS2), bornite (Cu5FeS4), enargite (Cu3AsS4), tetrahedrite (Cu12Sb4S13), tennantite (Cu12As4S13), covellite (CuS), chalcocite (Cu₂S), copper sulfide of the formula CuₓS_{y} wherein an atomic ratio of x:y is about 1 to about 2, carrolite (CuCo₂S₄), or combinations thereof, and wherein the non-copper bearing ore comprises millenite (NiS), pentlandite (Fe9Ni9S16), molybdenite (MoS2), violarite (FeNi2S4), cobaltite (CoAsS), pyrite (FeS2), linnaeite (Co3S4), sphalerite ((Zn,Fe)S), cattierite (CoS2), or combinations thereof.

17. (canceled)

18. (canceled)

19. The leaching solution of claim 1, comprising the leaching additive in an amount of about 0.001 mM to about 67 mM based on the total volume of the leaching solution.

20. (canceled)

21. The leaching solution of claim 9, wherein the metal values comprise copper, cobalt, nickel, zinc, molybdenum, vanadium, or combinations thereof.

22. A method of recovering metal values from an ore comprising metal sulfide species, the method comprising:
contacting and leaching the ore with a leaching solution comprising an organic compound comprising a sp³ hybridized sulfur, a sulfoxide, or a combination thereof and a lixiviant to form a pregnant leaching solution;
extracting metal values from the pregnant leaching solution using a solvent extraction process, wherein the solvent extraction process forms a metal-rich organic stream; and
recovering metal values from the metal-rich organic stream, optionally wherein recovering metal values is by an electrowinning process.

23. The method of claim 22, wherein leaching the ore is by in-situ leaching, heap leaching, percolation leaching, agitation leaching, or combinations thereof.

24. The method of claim 22, further comprising preparing a concentrate of the ore using a flotation technique prior to leaching, or using a smelter process to produce a matte prior to leaching.

25. (canceled)

26. The method of claim 24, wherein the matte is a nickel-copper matte or a lead-zinc matte.
